(19) United States
(12) Patent Application Publication
(10) Pub. No.: US 2010/0056391 A1
(43) Pub. Date: Mar. 4, 2010

(54) INTEGRATED SCREENING ASSAYS AND METHODS OF USE

(75) Inventors: Edward C. Cox, Princeton, NJ (US); Jeffry B. Stock, Princeton, NJ (US); Ying Chen, Princeton, NJ (US); Kyle J. McQuade, Bordentown, NJ (US); Satoshi Sawai, Tokyo (JP)

Correspondence Address: VENABLE LLP, P.O. BOX 34385, WASHINGTON, DC, 20043-9998, US

(73) Assignee: Trustees of Princeton University, Princeton, NJ (US)

(21) Appl. No.: 12/520,775

(22) PCT Filed: Dec. 20, 2007
(86) PCT No.: PCT/US07/25967

§ 371 Date: Jun. 22, 2009

Related U.S. Application Data
(60) Provisional application No. 60/876,481, filed on Dec. 22, 2006.

(51) Int. Cl. C40B 30/06; C40B 60/12; C12N 1/00

(52) U.S. Cl. 506/10; 506/39; 435/243

(57) ABSTRACT

A method and system is provided for screening compounds for biological activity in cultures of Dictyostelium.

### GOVERNMENT RIGHTS

This invention was made in part with United States government support under grant numbers NIH/NIGMS (R01 GM063677) and NIH/NICHD (PO1 HD39691). The government may have certain rights in the invention.

### FIELD OF THE INVENTION

The present invention is directed to screening assay systems, methods, and kits that can use Dictyostelium cells.

### BACKGROUND INFORMATION

Traditional models of drug development can be very costly and produce only a few, if any, viable compounds for clinical testing. These models generally involve separate assays to measure, for example, the efficacy, toxicity, and in vivo results of a candidate therapeutic compound. This approach can be effective but could be improved by developing drug screening systems capable of monitoring a more integrated cellular response than the present assays provide.

Current work to produce systems capable of monitoring the integrated cellular response improves on the classic experimental paradigm in developmental biology that begins with a mutant phenotype and then asks which aspects of development are altered. The goal is to relate structure to function, first at the molecular, then the cellular, and finally, the whole organism level.

This classical approach has been successful but, with the explosion of genome sequences, it is becoming realistic to rapidly map out relations between genotype and molecular level phenotype using large-scale assays at the level of transcription and translation. Efforts to complement such bottom-up approaches by high-throughput screens based on observational phenotypes at the cellular level have recently been reported in yeast, nematode and tissue culture cells. Friedman, A. & Perrimon, N. Genome-wide high-throughput screens in functional genomics, Curr Opin Genet Dev 14, 470-6 (2004). These studies have largely concentrated on the analyses of cell growth, division, and morphology, either through a growth curve analysis of batch cultures or by the analysis of morphology at a single to the few cell level by microscopy. See Hartman, J. L.t. & Tippery, N. P., Systematic quantification of gene interactions by phenotypic array analysis, Genome Biol 5, R49 (2004); Weiss, A., Delproposto, J. & Giroux, C. N., High-throughput phenotypic profiling of gene-environment interactions by quantitative growth curve analysis in Saccharomyces cerevisiae, Anal Biochem 327, 23-34 (2004); Harada, J. N. et al., Identification of novel mammalian growth regulatory factors by genome-scale quantitative image analysis, Genome Res 15, 1136-44 (2005); Ohya, Y. et al., High-dimensional and large-scale phenotyping of yeast mutants, Proc Natl Acad Sci U S A 102, 19015-20 (2005); Gonczy, P. et al., Functional genomic analysis of cell division in C. elegans using RNAi of genes on chromosome III, Nature 408, 331-6 (2000); Sonnichsen, B. et al., Full-genome RNAi profiling of early embryogenesis in Caenorhabditis elegans, Nature 434, 462-9 (2005); Neumann, B. et al., High-throughput RNAi screening by time-lapse imaging of live human cells, Nat Methods 3, 385-90 (2006). However, a comparable approach for a multicellular system based on quantitative real-time dynamical data gathered throughout the entire life cycle remains largely undeveloped.

Spatially and temporally evolving collective dynamics act critically to coordinate multicellular development. In general, periodic phenomena are prevalent in transcriptional regulation—for example, in circadian rhythms (Ueda, H. R. et al., System-level identification of transcriptional circuits underlying mammalian circadian clocks, Nat Genet 37, 187-92 (2005)), Msn transcription factor regulation in yeast (Jacquet, M. et al., Oscillatory nucleocytoplasmic shuttling of the general stress response transcriptional activators Msn2 and Msn4 in Saccharomyces cerevisiae, J Cell Biol 161, 497-505 (2003)) and the pulsatile response of NF-κB and p53 in tissue culture cells following stimulation (Nelson, D. E. et al., Oscillations in NF-kappaB signaling control the dynamics of gene expression, Science 306, 704-8 (2004); Lahav, G. et al., Dynamics of the p53-Mdm2 feedback loop in individual cells, Nat Genet 36, 147-50 (2004)). Oscillations seem to be a universal mode of regulation for morphogenetic cell movements and gene transcription that requires fine spatial and temporal coordination. Calcium waves are observed during convergent extension in Xenopus and are believed to coordinate cell movement. Wallingford, J. B. et al., Calcium signaling during convergent extension in Xenopus, Curr Biol 11, 652-61 (2001). In the case of somitogenesis, where segmentation is periodic, Notch and Wnt signaling is coupled to periodic expression of the Notch components themselves. Horikawa, K., Ishimatsu, K., Yoshimoto, E., Kondo, S. & Takeda, H., Noise-resistant and synchronized oscillation of the segmentation clock. Nature 441, 719-23 (2006); Masamizu, Y. et al., Real-time imaging of the somite segmentation clock: revelation of unstable oscillators in the individual presomitic mesoderm cells. Proc Natl Acad Sci U S A 103, 1313-8 (2006).

Functions of molecular networks may become apparent only when put into the context of such multicellular organization in time and space. Biologically relevant readouts with a temporal and spatial resolution can connect high-throughput genomics data obtained at the molecular and cellular level to higher organizational and functional levels. These biological readouts of the integrated response of cells, including the spatiotemporal characteristics of cells, can be used in a system of screening candidate therapeutic compounds for efficacy capable of filling the need in the art for improved methods of drug screening.

### SUMMARY

Some embodiments include a system for screening test compounds for biological activity, the system comprising: at least one culture of Dictyostelium cells or an array of Dictyostelium cells; a material handling device that contacts cultures or the array with the test compounds, an array handling device that positions the arrays of cultures at predetermined locations, an automated image capture device that captures images of the cultures; and a data processor that process the images and outputs at least one quantitative measurement of the response of the cultures to the test compound.

Some embodiments include a method for screening a plurality of test compounds for biological activity, the method comprising: preparing an array of cultures of Dictyostelium cells; contacting cultures with test compounds; capturing images of the cultures using an automated image capture device; processing the captured images to quantitate a characteristic of the cultures; and determining from the quantitative culture characteristic and a control value whether the addition of the test compound resulted in a biological integrated response of the culture.

Some embodiments include a method for screening a compound that is exogenous to Dictyostelium for biological activity, the method comprising: preparing a culture of Dictyostelium cells; adding the compound to the culture; using an automated image capture device to capture images of the culture; and comparing the spatial-temporal properties of the culture to control images to determine the biological activity of the compound.

Some embodiments include a method for screening a compound that is exogenous to Dictyostelium for biological activity, the method comprising: preparing a culture of Dictyostelium cells; adding the compound to the culture; using an automated image capture device to capture images of the culture; and comparing the captured images to control images to determine the temporal properties of the culture to determine the biological activity of the compound.

Some embodiments include a method for screening a plurality of compounds for biological activity, the method comprising: preparing a plurality of cultures of Dictyostelium cells; adding the compounds to the cultures; using an automated image capture device to capture images of the cultures; and comparing the captured images to control images to determine the phenotypic changes of the cultures to determine the biological activity of the compounds.

Some embodiments include a method for generating a database of Dictyostelium images, the method comprising: (a) preparing clonal populations of Dictyostelium in parallel, (b) recording images of the life cycle of the populations in a computer readable format using an automated image capture device; and (c) storing the images on a computer to generate a database.

Some embodiments include a kit for screening a test compound for biological activity, the kit comprising: Dictyostelium cells or spores, selected from wild type and mutant types having a known integrated response to a predetermined category of compounds, when grown in culture and contacted with a compound in the predetermined category, as detected in an automated image capture device.

Some embodiments include a computer readable data structure, encoded on a computer readable medium, the structure comprising data records, each of which is correlated to a Dictyostelium culture and imaging data associated with the culture, wherein at least one of the data records for a Dictyostelium culture further comprises data identifying a test compound with which the culture has been contacted.

Some embodiments include a method for characterizing the developmental stages of Dictyostelium comprising: observing in at least one Dicytostelium cell the deviation from wild-type behavior in at least one of the following developmental stages: growth, early wave, aggregation, mound, slug and fruiting body; ranking the observed cell into at least four different categories pᵢⱼ=−1, −½, 0, ½ or 1, wherein i ∈ [1, 6] stands for ordered developmental stage and j represents the sample number, and assigning a phenotypic score to the observed cell.

Some embodiments include a method for testing a plurality of compounds for biological activity comprising the steps of: providing an array comprising a plurality of Dictyostelium cultures; placing each of the compounds into contact with at least one of the cultures; determining the location of each compound and culture in the array; capturing multiple images of each culture over a period of time; processing the images to quantitate characteristics of the cultures; and determining whether one or more of the compounds causes a visible biological response.

Some embodiments include a system for screening test compounds for biological activity, the system comprising: an array of cultures of cells; a material handling device that contacts cultures with the test compounds, an array handling device that positions the arrays of cultures at predetermined locations, an automated image capture device that captures images of the cultures over time; and a data processor that process the images and outputs at least one spatiotemporal measurement of the response of the cultures to the test compound.

### DESCRIPTION OF THE DRAWINGS

FIGS. 1(a)-(d) illustrates an embodiment of the automated image acquisition and phenotyping—of clonal populations that can be used in the present invention. (a) Illustrates the flow chart for assaying over 2000 insertional mutant clones that were subjected to parallel culture and phenotyping as described herein. (b) Illustrates the gantry robotic system that can be used in some embodiments of the present invention. The darkfield optics can be positioned below the samples, while the digital camera can be above. (c) Contains snapshots of movies from wild type AX4 cells at representative stages in development. Images were captured every 40 sec from each well for 10.5 hrs after plating for a total of 800 frames/well. Later stages of morphogenesis were then followed for 28.5 hr by bright-field illumination. During this period, images were captured at 127 sec intervals, also for a total of 800 frames from each well. The images in the first column were obtained from a 16.8 mm×12.6 mm area by averaging 5 frames taken approximately 66 msec apart for noise reduction. Successive averaged frames were then subtracted to obtain the wave images in the second column (3 and 5 hr). Bright-field optics were employed for the second half of the imaging session to follow slug motion (11 hr). After the run was over, the final culminant morphology was checked under a dissecting microscope (48 hr). (d) Illustrates the wavelet portrait. For the first 10.5 hr, frequency data were obtained from averaged wavelet transformations of pixel intensities as a function of time (see main text for details). Wavelet power spectrum is color coded, and the slow increase in frequency, then abrupt termination, followed by long-period features caused by cell streaming and territory formation, are indicated with arrows.

FIGS. 2(a)-(e) illustrates phenotypic clustering based on the timing of mutant behavior. (a) Illustrates the results of the 2257 strains that were assigned phenotype vectors according to the stage-specific mutant defect. A correlation coefficient was used as the phenotype similarity metric. Average linkage clustering was performed on qₛⱼ with zero offset. (b) Illustrates an expanded view of developmentally-null and other severely impaired mutant clusters. (c) Illustrates a mid- to late-stage development mutant cluster. The table on the right side lists the corresponding V-strain ID's in addition to the dictyBase ID and gene name of the disrupted locus.

FIGS. 3(a)-(i) illustrates early cell-cell signaling. (a) Illustrates the results obtained when a wavelet transform was further reduced to a one-dimensional representation by tracing the peak of the averaged wavelet power spectrum as a function of time t. The traced data were then subjected to K-mean clustering. The bottom cluster comes from experimental runs where the normal 5-min optical-density oscillations were not detected. Other clusters are wild type with respect to signaling periodicity but are grouped according to the difference in wave onset. The second bottom cluster shows large deviations in the timing and consists mainly of low cell density samples. (b) Illustrates the frequency of the optical density oscillations before termination is narrowly distributed and highly reproducible. (c) Illustrates that a wavelet power spectrum on the other hand follows log-normal distribution. (d) Illustrates that the number of spiral cores per 2.1 cm² area and (e) the time of cessation of the periodic signaling follows a Gaussian distribution (shown in dotted curves). (f-i) Illustrates that scatter plots indicate relations between these measures that reflect properties of the self-organizing pattern formation from random initial conditions (see main text for details). Correlation coefficients are (f) −0.20, (g) 0.05, (h) −0.37 and (i) 0.15 respectively.

FIGS. 4(a)-(d) illustrates representative samples with defects in early development. The severity of the signaling phenotype ranges from the absence of optical-density waves to delayed slow oscillations. Frame-subtracted images at t=6-8 hr (left) and the original images at t˜10 hr (center). Wavelet portraits are shown on the right. (a) V10233 (piaA) shows no sign of periodic signaling. (b) V10285 (DG1105) shows local pulsatile activity while (c) V10199 (DG1037) and (d) V10682 (clcD) are slow oscillators with incomplete aggregation or delayed aggregation, respectively.

FIGS. 5(a)-(c) illustrates that the screen identifies mutants with altered development. Frame-subtracted images at t=2-4 hr (left) and the raw images at t=5-8 hr (center). Wavelet portraits are shown on the right. (a) Wild type AX4. (b) V30230 (regA) and (c) V10258 (rdeA). The signaling period is emphasized by the red lines above each portrait.

FIGS. 6(a)-(c) illustrates that the screen uncovers mutants with aberrant slug motion. The multicellular slug phenotype is often difficult to see in cells feeding on bacterial lawns (left) because development is asynchronous and the slug stage is transient. The middle panels are snapshots from our automated imaging system taken at ˜24 hr. Slug trajectories over a 28.5 hr period were obtained by first binary thresholding the movies and then tracking the center of mass by multiple particle tracking using ImageJ (right hand panel).

FIG. 7 illustrates the development of Dictyostelium cells at 6.5, 8.5 and 9.25 hours in the presence of an inhibitor of signaling (AFC), an inactive analogue of the inhibitor (AGC), or with no treatment (control).

### DETAILED DESCRIPTION

Embodiments of the present invention are directed to systems, methods, and kits that can use Dictyostelium (slime mold) cultures in drug screening assays. The cultures prepared for use in the various embodiments of the present invention can be wild type or mutant cells. In some embodiments, the culture of Dictyostelium can be a culture of wild type Dictyostelium, a culture of mutant Dictyostelium, or combinations thereof.

The culture of Dictyostelium cells can be a mutant selected for any desired phenotype or genotype. A wide variety of Dictyostelium mutants capable of being used in the present invention can be found at DictyBase, available at dictybase.org.

This website includes numerous movies further illustrating the results of some aspects of the present invention, including wild type and mutant cultures, with some results presented on a gene-by-gene basis. Additional examples and full description of these methods and analyses may be found in Sawai, S., Guan, X.-J., Kuspa, A., and Cox, E. C. (2007). High-throughput analysis of spatio-temporal dynamics in cell populations. Genome Biol. 8, R144, pp 1-15. (cover photo, and see “My 2,000 best films: parallel phenotyping of Dictyostelium development,” G. Bloomfield and R. Kay, Genome Biology 2007, 8(7):220). The additional video clips of mutant and wild type strains analyzed by the techniques described herein are linked to the genomic sequence and may be queried by going to “dictybase.org/phenotype/movies/index_dictybase.php”. The sources are incorporated by reference in their entirety, including for the various mutant cultures and color and black and white images included therein.

In some embodiments, the system comprises more than one culture of Dictyostelium cells. The cultures can be of a uniform phenotype and/or genotype or the cultures can have different phenotypes and/or genotypes. In some embodiments, the system for screening compounds for biological activity comprises more than one culture of mutant Dictyostelium cells, more than 5 cultures of mutant Dictyostelium cells, more than 10 cultures of mutant Dictyostelium cells, more than 15 of mutant Dictyostelium cells, or more than 20 cultures of mutant Dictyostelium cells.

In some embodiments, the systems of the present invention can comprise at least one culture of mutant Dictyostelium cells and at least one culture of wild type Dictyostelium cells.

In some embodiments, the mutant Dictyostelium cells have altered periodic cAMP oscillations. There are two major events required for periodic cAMP oscillations. One is the activation of adenylyl cyclase upon cAMP binding to the receptor, which will raise the level of cAMP. A rise in cAMP is also facilitated by the inhibition of the intracellular phosphodiesterase, RegA, via Erk2. The other is the adaptation of adenylyl cyclase, which stops the production of cAMP, thereby allowing the cAMP level to come down by intracellular and extracellular diffusion and degradation.

Compared to the two activation pathways that are relatively well known, the molecular mechanism(s) responsible for adaptation are largely unknown. Adaptation may occur in response to continuous exposure of the cell to either 1) high extracellular cAMP levels or 2) high intracellular levels. In model 1), it is thought that high extracellular cAMP levels will change the state of the receptor (receptor phosphorylation serves as a good indicator of adenylyl cyclase adaptation), and turns on a specific adaptation pathway that brings adenylyl cyclase to an inactive state. The extracellular cAMP level will come down due to extracellular phosphodiesterase, and the resulting reduced occupancy of the receptor allows it to ‘de-adapt’ and be ready for the next activation. In model 2), high intracellular cAMP levels increase PKA activity and thereby either inhibit adenylyl cyclase directly, or indirectly by changing the receptor state. In this scenario, the receptor de-adapts because the intracellular phosphodiesterase RegA is soon reactivated, because MAP-kinase Erk2 activation (which inhibits RegA) is only transient.

In some embodiments, an assay may involve determining whether a test compound causes a wild type culture to exhibit an integrated response that is characteristic of a particular mutant phenotype, such as a spatiotemporal phenotype. In some embodiments, an assay may involve determining whether a test compound causes a mutant culture to exhibit an integrated response that is characteristic of a wild type phenotype, such as a spatiotemporal phenotype.

In some embodiments, a mutant cell culture used in the systems for screening a compound for biological activity can be selected for a particular spatiotemporal phenotype. The assay may involve determining whether a test compound causes an integrated response such as reverting the mutant phenotype back to a wild type phenotype.

Some examples of mutants having particular phenotypes which may be used in determining an integrated response to a compound according to the invention are as follows: early wave mutants, pulse mutants, slow oscillator mutants, amplitude mutants, PKA pathway mutants, slug mutants, and combinations thereof.

In some embodiments, the mutant can exhibit an early wave phenotype. An “early wave phenotype” means the excitable state and wave propagation beginning within a few minutes after its onset.

Whether a mutant has an early wave phenotype can be determined by examining several wavelet parameters that characterize early cAMP signaling. Characteristic wavelet parameters are frequency, amplitude and time of onset. An example of this technique is provided in Example 3. Other approaches will be apparent to one of ordinary skill in the art.

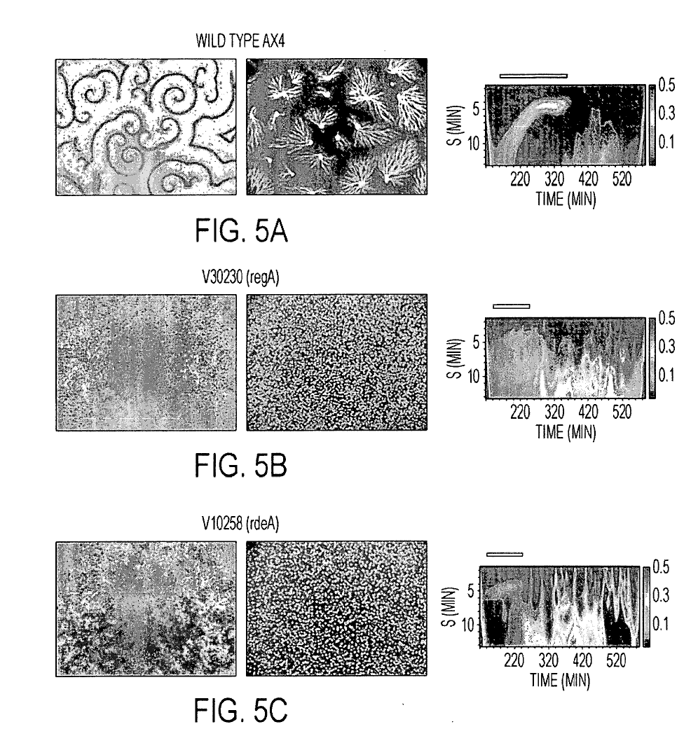

The Dictyostelium culture used in the present invention can also comprise pulsing and slow-oscillator mutants. Pulsing mutants are shown in FIG. 5b, c. Slow oscillator mutants exhibit oscillating spiral cores that oscillate at periods longer than wild-type. A pulsing or slow-oscillator mutant can be identified by searching for those that failed to exhibit the typical developmental time-course in optical density oscillations. An example of this technique is provided in Example 4. Other approaches will be apparent to one of ordinary skill in the art.

The cultures of the present invention can also comprise an optical-density wave mutants. “Optical-density wave” mutants have waves that fail to propagate at normal velocities amplitudes or waves that break up or die out.

The cultures of the present invention can also comprise a PKA pathway mutant. A “PKA pathway mutant” is a strain that is mutated in one or more proteins that serve to control the activity of PKA including, for example, adenylyl cyclase, cAMP phosphodiesterase or any protein that directly or indirectly regulates either of these two activities, or mutations in the PKA regulatory or catalytic domains, or mutations in any other protein kinase or phosphoprotein phosphatase that regulates any of these components, or mutations in DNA regulatory regions that alter levels of expression of any of these components, or mutations in transcription factors or in genes that directly or indirectly regulate transcription factors that control levels of expression of any of these components An example of this technique is provided in Example 5. Other approaches will be apparent to one of ordinary skill in the art.

The present invention can also comprise a slug mutant. A “slug mutant” refers to a Dictyostelium cell that has an alteration that affects its ability to form a slug or its behavior as a slug.

In some embodiments, the present invention is directed to monitoring the development of slugs using mutant cells. In Dictyostelium, a slug is a multicellular structure consisting of anterior prestalk cells and posterior prespore cells that migrates towards favorable environments for culmination. Studies suggest that propagating waves of cAMP not only direct cell aggregation during the early stage of development, but also coordinate cell migration in the slug stage. Dormann, D. & Weijer, C. J. Propagating chemoattractant waves coordinate periodic cell movement in Dictyostelium slugs, Development 128, 4535-4543 (2001); Bedford, M. T. & Richard, S., Arginine methylation an emerging regulator of protein function, Mol Cell 18, 263-72 (2005). Slug migration velocity is typically of the order of several hundred microns per minute, and therefore its characterization is difficult without time-lapse imaging. An example of monitoring the development of slug mutants is provided in Example 6. Other methods for analyzing the coordinated behavior of slug mutants will be apparent to a person of ordinary skill

As one of skill in the art will appreciate, mutants other than those listed herein can be selected for use in some embodiments of the present invention. Thus, the present invention is not limited to the mutants described herein.

The systems of the present invention can be used to screen a wide variety of compounds for biological activity. As one of skill in the art will appreciate, any compound capable of causing or changing an integrated biological response in Dictyostelium can be used in the present invention.

In some embodiments, the compound is selected from the group consisting of an inorganic molecule, an organic molecule, a drug, prodrug, antibody, vaccine, nucleotide, polynucleotide, amino acid, peptide, polypeptide, virus, and combinations thereof.

Some non-limiting examples of compounds that can be adapted for use in the present invention include: agonists and antagonists of G protein-coupled intracellular and extracellular signaling; botanical extracts to screen for biologically active agents (e.g. coffee extracts, chocolate, ginkosides, cannabinoids, etc.); anti-folate agents used to treat cancer and arthritis such as methotrexate; anti-cancer chemotherapeutic agents that target microtubules such as taxol; agents such as valproic acid that target the phosphatidylinositol signaling system and are used to treat bipolar disorders; agents such as rapamycin that target TOR signaling pathways; agents that generally target G-protein mediated inflammatory responses such as N-acetyl-farnsyl cysteine (AFC); agents that target protein kinase and/or phosphoprotein phosphatase regulatory systems; and agents that inhibit the types of ABC export systems that lead to the resistance of some cancers to chemotherapeutic agents.

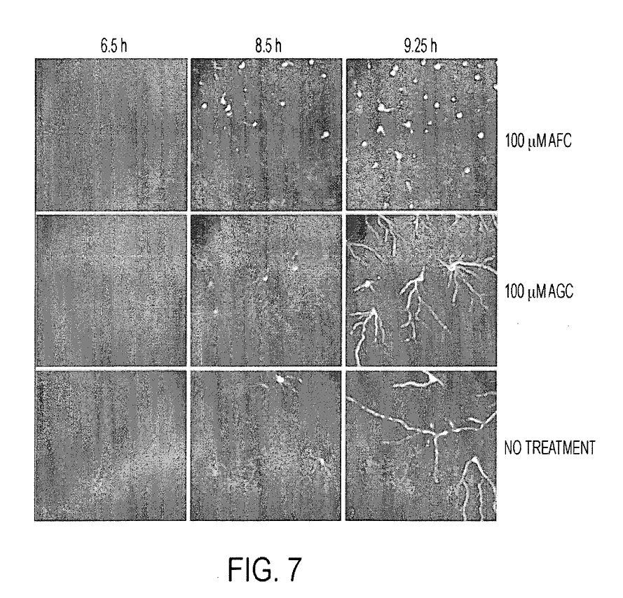

For example, as illustrated in FIG. 7, the effects of AFC on Dictyostelium cells have been monitored. These experiments demonstrated that AFC can alter the mechanism by which individual cells aggregate into multicellular clusters. Untreated cells line up head-to-tail and form streams while migrating while cells treated with AFC migrate, but do not line up head-to-tail. AFC also inhibited the ability of the cells to form fruiting bodies. Accordingly, some embodiments of the present invention are directed to assays testing the effects of AFC on Dictyostelium cells.

In some embodiments, the compound used in the present invention can be a non-endogenous compound. A non-endogenous compound is a compound that is not found in wild type Dictyostelium cells. Non-endogenous compounds do not include signaling molecules, peptides, and nucleotides normally found within wild type Dictyostelium. For example, cAMP would not be considered a non-endogenous compound because it is a signaling molecule found in wild type Dictyostelium.

In some embodiments, the compound used in the present invention can be an endogenous compound that may have utility as a pharmaceutical agent. An endogenous compound is a compound that is found in wild type Dictyostelium. Endogenous compounds include signaling molecules, peptides, and nucleotides normally found within wild type Dictyostelium. When an endogenous compound is used as a test compound, it can be used at a different concentration from that found in wild type cells, and in mutant cells, to determine whether a higher or lower concentration may have a desirable activity.

In some embodiments, the compound used in the present invention can be a compound with a known effect when it contacts a Dictyostelium cell or a culture of Dictyostelium cells. In some embodiments, the compound used in the present invention can be a compound with an unknown effect when it contacts a Dictyostelium cell or a culture of Dictyostelium cells.

In some embodiments, the Dictyostelium cells used in the present invention can be altered by mutation or otherwise to increase the permeability of the compound into the Dictyostelium cell.

In some embodiments, the Dictyostelium cell can be altered to increase the retention of the compound within Dictyostelium once it has permeated the cell. Thus, a strength of the present system is that Dictyostelium can be manipulated genetically using the various techniques know to one of skill in the art. It is known, for example, that there are many ABC transporters in the Dictyostelium genome, and these can be expected to export some test compounds with enough vigor to make the test meaningless. This is a common problem with mammalian and other cell lines. In the case of Dictyostelium, however, it is straightforward, using the Cre/Lox system, to delete any number of ABC transporters in any permutation or combination, thereby sensitizing the genetic background to ever wider panels of test compounds. This is useful when a new compound is discovered that has a modest effect on a particular pathway, because members of the pathway can then be engineered to make the test more sensitive, and thus capable of uncovering additional hits from a library.

For high throughput use according to the invention, cultures of Dictyostelium can be prepared as an array of cultures in multi-well configurations, such as those readily available (e.g. 398 or 96 well plates, or plates with lower numbers of larger wells, such as 6 or 9 well plates). The culture plates or arrays need to be suitable for automated handling, and with optical characteristics susceptible to imaging. By arraying cultures of the same or different genotypes of Dictyostelium, the scientist may test different compounds on different cultures of the same genotype, different concentrations of a compound on different cultures of the same genotype, or the same compound on cultures of different genotypes of Dictyostelium, and other analytical models that would be readily apparent to a person of ordinary skill in the art.

In some embodiments, the present invention comprises an automated image capture device. An “automated image capture device” is a device capable of collecting images until it is stopped or a specified event is reached, without the need for human assistance.

In some embodiments, the automated image capture device used in the present invention comprises a robot that has been adapted to capture images of the Dictyostelium cultures. Suitable ways to adapt a robot for use in the present invention include attaching a camera to it, attaching dark field illumination optics to it, and/or attaching a fluorescent microscope to it. See Sawai et al., An autoregulatory circuit for long-range self-organization in Dictyostelium cell populations, Nature 433:323-326 (2005). In some embodiments, the automated image capture device comprises a lens capable of magnifying the image of the Dictyostelium cultures.

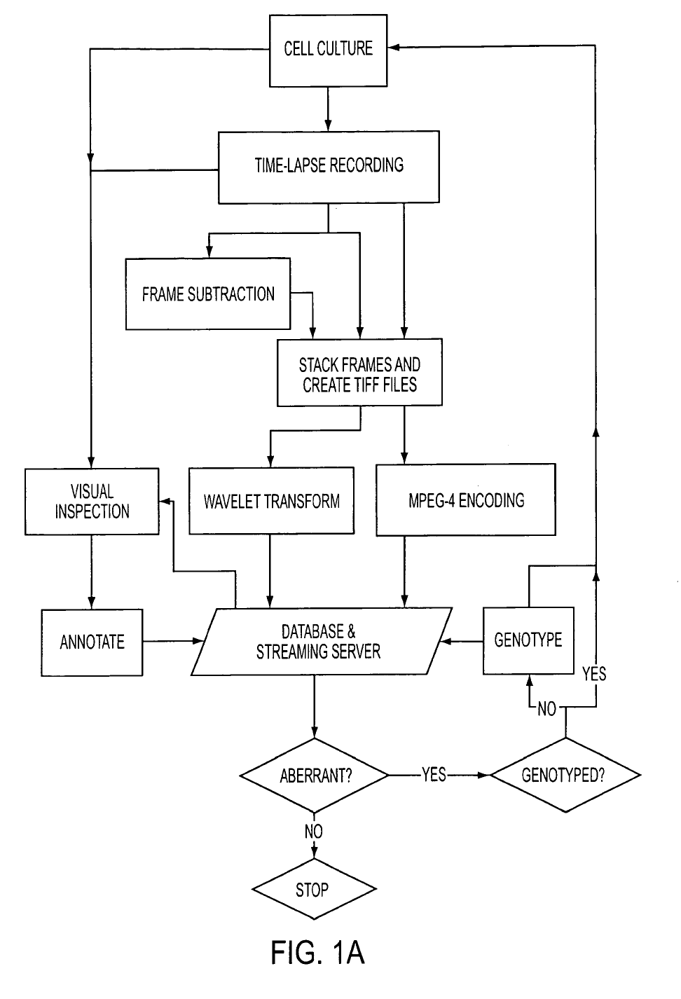

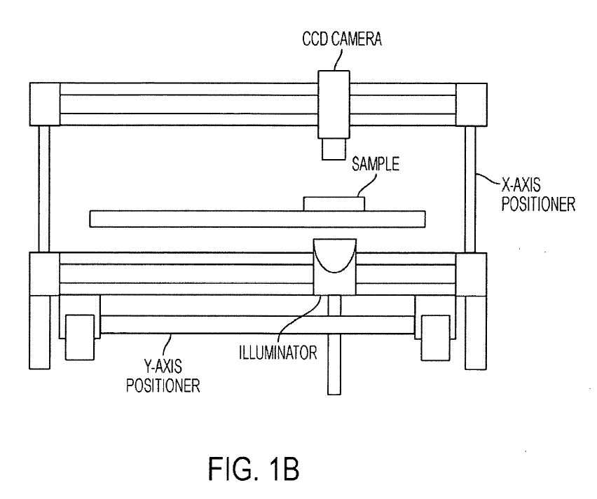

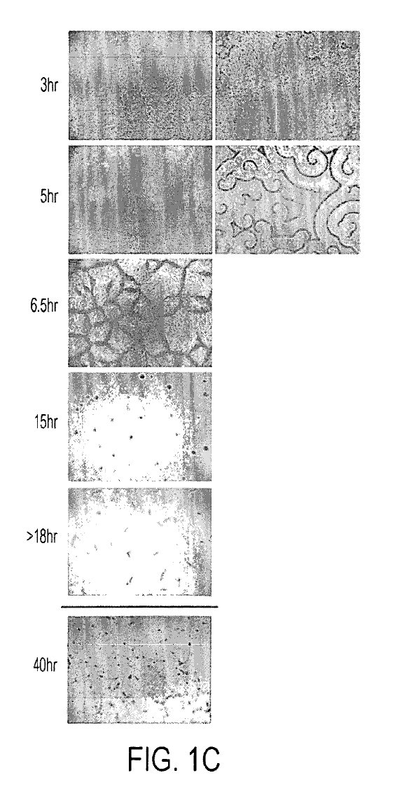

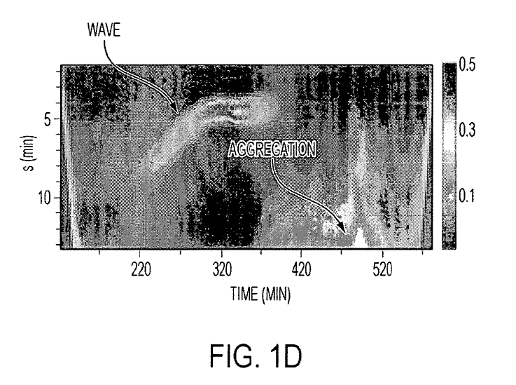

One non-limiting example of a suitable image capture device used in the present invention is an imaging robot that was constructed using industrial automation assemblies as a gantry system, with two x-y instrument platforms ganged together, one positioned above the sample holding area, the other below, each driven by digital servo drives (Gemini GV; Parker Automation) (FIG. 1b). The drives were operated through a programmable two-axis servo controller (6K2; Parker Automation). The servo tuning and axis-control programs were written using Motion Planner software (Parker Automation). The upper gantry platform housed a ⅓ inch format CCD camera (LCL-903HS; Watec) with a macro lens. The dark-field illumination optics consisting of a fiber optics light guide and lenses, is mounted on the lower platform. Although this is a belt-driven system, feed-back loops in the controllers allowed positioning over the 2×2 meter sample platform with a reproducibility of ˜100 μm rms. Time interval fluctuation measured at a single well for both the first and second time intervals was typically 0.1 sec (standard deviation). The robot can be housed in a light-tight room at a constant temperature of 22° C.

The robot can comprise a fluorescent microscope. The fluorescent microscope can be used to capture images of fluorescently tagged proteins to monitor protein localization. In this embodiment, the illumination source would replace the dark-field optics in FIG. 1b, and the fluorescent microscope optics of the camera.

In some embodiments, the robot of the image capture device can be controlled using software. The software can have several functions:

(1). The software can allow a person to program all of the necessary features for reliable robotic runs, including (in both the x and y axes), acceleration and velocity, soft limits to travel, accuracy, dwell time at each location, and feed-back and feed-forward loops that essentially correct for position. The x and y axis drivers can also supply TTL signals that are used to trigger e.g. the camera shutter, so that image capture is synchronized with x,y position. An example of this type of software is available from Parker automation (“Motion Planner”).

(2). The software can grab image frames to create an image stack. When the robot stops at each location, n video frames are grabbed by a Scion frame grabbing board, averaged on the board, and written to a RAID hard drive where they are then processed. The frame grabbing software is supplied by Scion and it is modified as needed using a macro language. Alternatively, a fire-wire connection and commercial or free-ware software can be used.

(3). The software can assist in processing the image stack, for example, by using NIH Image J. The signal is enhanced by subtracting averaged frames, and the frames are stacked as a video clip. See Supplementary videos to Sawai et al., An autoregulatory circuit for long-range self-organization in Dictyostelium cell populations, Nature 433:323-326 (2005), available at www.nature.com/nature/journal/v433/n7023/suppinfo/nature03228.html

The image stack is then processed in two ways: first, the entire video record is analyzed with a wavelet function and converted into a wavelet portrait which summarizes wave frequency and amplitude vs. time. The number of spiral cores, which is one measure of the excitability of the system, is computed using an algorithm similar to one published by Gray et al. Nature 392, 75 (1998). Both analyses have been automated.

The automated image capture device can be used to generate a database of the spatiotemporal response of cells using high-throughput techniques. Accordingly, the present invention, in some embodiments, is directed to a method for generating a database of Dictyostelium images, the method comprising: (a) preparing clonal populations of Dictyostelium in parallel, (b) recording images of the life cycle of the populations in a computer readable format using an automated image capture device; (c) storing the images on a computer-readable medium as a database; (d) processing the images by wavelet function analysis to produce a wavelet portrait, and/or calculating the number of spiral cores; and (e) storing the processed image data on a computer readable medium as a database.

For example, six-well plates were placed on a stage that can hold up to one hundred accurately aligned in the x-y plane. Images from a 16.8 mm×12.6 mm area from each well were captured and transferred to a computer, where they were digitized and stored in 640 by 480 pixel 8-bit grayscale TIFF format using a frame grabbing board (LG-3; Scion Corporation). Image files were written to a high capacity hard-disk system (Xserve RAID; Apple Computer).

Image acquisition, frame stacking and frame subtraction were accomplished using Java-based plug-in applications written for ImageJ (available at rsb.info.nih.gov/ij/). These files were encoded in MPEG-4 format using ImageJ and Quicktime Pro (Apple Computer) for easy viewing over the Internet using a streaming server. Subtracted movie files were encoded at 12 frames per second. The first and second-half of the original movies were encoded at 48 and 36 frame per second, respectively. Movie files, wavelet data and annotation data were stored on a MySQL server. Data acquisition, data management and statistical analyses using the MySQL database were performed with web-based queries written in PHP and the R statistical package (available at www.R-project.org).

Embodiments of the invention include electronic and electromechanical and optical hardware such as the optical imaging and sample handling systems, subsystems, and components, and computational and statistical tools and methods, steps, and subroutines. These include tools and methods used for collecting, storing, analyzing, and retrieving data, comparing spatiotemporal characteristics for test compounds and controls, data mining, and data visualization; pattern recognition tools; and predictive tools. User interfaces are encompassed within the invention.

The inventive methods and systems, including subsystems, steps, and routines, may be practiced by an individual or group of individuals working in a single research laboratory. Alternatively, the inventive systems and methods may be used, in part, in a distributed computing “system”, having a client and a server side. For example, the data collection steps may be practiced in a laboratory, from which the data is sent to a remote server where the data is processed, analyzed, and stored. The output from the server side may be a complete data transfer sent back to the laboratory where the data is collected, or it may be a conclusory or summary output, such as an indication of which test compounds out of a library were determined to be leads for further analysis in relation to a particular biological target.

Embodiments of the invention include data compiled or assembled using the system, and methods for compiling and assembling the information, as well as data structures produced by the methods and systems of the invention.

An exemplary apparatus according to the invention permits automated assays of test compounds in Dictyostelium cultures positioned in a plurality of wells or plates in an array or culture holding device that is suitable for automated handling. Such an apparatus may include automated optical imaging means for viewing the cultures at each of a plurality of array locations and for producing digital images corresponding to locations at and within the cultures, and means for storing the digital images of the cultures, and means for producing time lapse videos of each culture. An exemplary apparatus may comprise means for holding the arrays, e.g. multi-well plates, and moving them, or the imaging device, in relation to each other with sufficient precision to produce time lapse images automatically. An apparatus of the invention may comprise a means for calibrating the device by automatically measuring at least one known measurable attribute of a calibration culture, which may serve as a control.

The data processing component of the inventive apparatus may include means for generating an automatic video signal from scanned images of the cultures, processing means for processing the automatic video signal for each of the cultures to output at least one measurable attribute value for the culture, wherein said processing means correlates the recorded images for each of the cultures, quantitates spatial and/or temporal characteristics of the cultures, and compares them to a characteristic of a control culture. The apparatus may also include means for determining which of the compounds, if any, meets the criteria for a lead compound having a desired biological activity, and provides output means for displaying images and data, such as raw images and processed data regarding each of the cultures.

The inventive apparatus may include means for storing calibration or control values, and for storing data for the test cultures (cultures contacted with test compounds), so that they may be compared. A plate handler according to the invention may be configured for automatically shifting an array to position the array at a predetermined location, e.g. at the stage of a microscope. The image capture assembly may include a microscope with suitable optics, which may be automatically focused, and a light source suitable to the microscope and the intended image capture procedure.

The present invention provides methods and systems for quantitatively measuring the integrated response of Dictyostelium cells in culture. The “integrated response” of cells in culture is a complex biological activity such as the spatial and/or temporal (spatiotemporal) properties exhibited by the cells in the culture, or the intercellular behavior of groups of cells in the culture. The integrated response may result from the function of more than one signaling pathway, otherwise referred to as a signal transduction pathway within a Dictyostelium cell. A “signal transduction pathway” is a sequence of linked interactions, for example between proteins, that serve to generate cellular responses to sensory inputs.

Signal transduction pathways begin with sensory receptors that interact directly with specific stimuli. The stimuli can be nutrients (sugars, amino acids, lipids, etc), signaling molecules from other cells (pheromones, hormones, cytokines, neurotransmitters, etc), drugs (morphine, caffeine, nicotine, etc), or any other agent or environmental perturbation that can cause a change in protein structure. A given stimulus-receptor interaction acts to modulate the subsequent interaction between the receptor and the next downstream component of the signal transduction pathway.

Some examples of signal transduction pathways which may be involved in an integrated response that can be measured according to the invention are as follows: a G protein coupled receptor pathway (e.g., the cAMP signaling pathway), a growth factor receptor pathway, or a membrane channel receptor pathway.

For instance, in the case of the cAMP signaling pathway in Dictyostelium, the cAMP receptor, cAR1, spans the cytoplasmic membrane. The inactive receptor-associated G-protein has a GDP bound to its alpha subunit, Ga2. When cAMP is released from a cell, it can bind to a specific site on cAR1 that is exposed to the external milieu. cAMP binding causes a change in receptor conformation that triggers the conversion of the GDP-bound form of Ga2 to its active GTP-bound form. This leads to the release of the G-protein from the receptor and the co-ordinate dissociation of the GTP-bound Ga2 subunit from Gbg. Ga2 goes on to interact with signaling pathways that serve to regulate motility and chemotaxis, while Gbg interacts with a pathway that serves to activate an enzyme, adenylyl cyclase, that catalyzes the further production of cAMP. The combined effect on multiple signal transduction pathways is the generation of waves of aggregating cells.

The cAMP receptor belongs to a large family of homologous membrane receptors termed GPCRs (G-protein coupled receptors), all of which initiate signal transduction pathways that depend on G-protein activation. There are more than 1000 members of the GPCR family encoded in the human genome including virtually all of the metabotropic neurotransmitter receptors in the brain.

Other common classes of receptors include the growth factor receptors and the membrane channel receptors. The former generally function to initiate signal transduction pathways through the activation of protein kinases. The latter activate pathways through changes in membrane potential. Although the mechanisms of pathway activation differ between different receptor classes, the principle remains the same.

Each receptor can interact with a distinct set of stimulatory inputs. Stimuli can be referred to as receptor modulators, and the type of modulation can generally be subdivided into two classes: agonists and antagonists. Agents like cAMP that lead to the activation of a signaling pathway are termed agonists. Often it is desirable to develop drugs that block the activation of a specific receptor. These are termed antagonists. Antagonists are often analogues of agonists that compete for binding but do not elicit a response. A classic example is the opiate receptor antagonist, naloxone, that specifically binds to the GPCRs that mediate responses to opiates and thereby blocks the effects of morphine. It is used clinically to treat individuals suffering from a heroin overdose.

According to the invention, a test compound may be identified as a modulator, e.g. an agonist or antagonist, of a particular receptor if it causes a particular integrated response that is characteristic of a modulator, e.g., an agonist or antagonist of that receptor. In a wild type culture, the test compound may change the culture's phenotype to be different than wild type. In a mutant culture, the test compound may change the culture's phenotype to be different from the mutant phenotype, e.g. more like, or the same as wild type.

In some embodiments, the integrated response is quantitatively measured in Dictyostelium by examining the spatiotemporal dynamics of cooperating cell populations in culture. For example, continuous time-lapse histories of over 2,000 mutant clones from a large-scale mutagenesis collection have been sampled to generate quantitative information. This can produce a record of the temporal and spatial dynamics of each mutant, including the onset and evolution of traveling cyclic AMP (cAMP) waves, the transition from stationary signaling cells to cells streaming toward an organizing center, and the motion of the multicellular slug as it forms a mature fruiting body.

In some embodiments, a coarse-grained phenotypic space for clustering mutants in the form of a ‘phenotypic array’ can be used. Approximately 4% of the clonal lines created in an unbiased forward screen were mutant at one stage in this array. Many of these, along with known mutants, can be ordered by hierarchical clustering into functional groups. Among the mutations identified were independent occurrences of known genes and new mutants in common phenotype clusters, and mutant phenotypes originating from intergenic insertions. The resulting dataset allows one to search and retrieve life cycle movies and analysis on a gene-by-gene and phenotype-by-phenotype basis. In some embodiments, this dataset can be used to determine the proper mutants for a culture of Dictyostelium cells capable of being used according to the present invention.

In some embodiments, phenotype clustering can be used to quantitatively group and measure the properties of mutant Dictyostelium cells. For example, about 1,800 insertional mutants (hereafter referred to as the unbiased set from an ongoing large-scale mutagenesis project (dictygenome.bcm.tmc.edu/)), and about 400 containing many previously isolated mutants have been sampled. In addition to the quantitative features described for the early developmental stages, qualitative features such as cell morphology during axenic growth, slug motion/morphology and fruiting body structure were obtained from the movies and observation of the samples by microscopy. From these features, a phenotype matrix pᵢⱼ was obtained. The matrix is a digital representation of whether or not strains exhibited aberrant behavior at each stage of development.

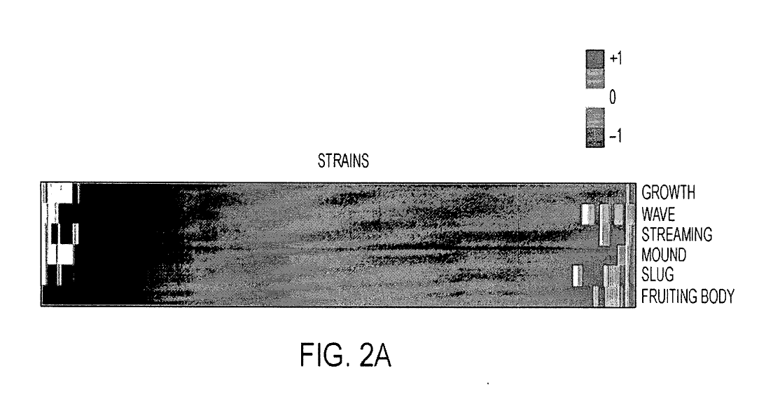

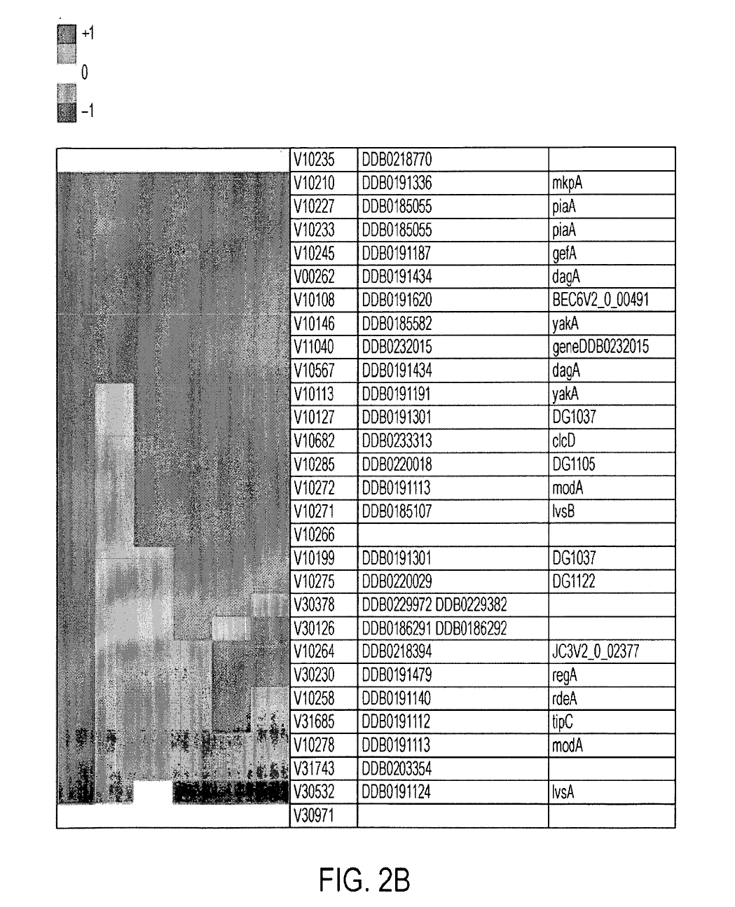

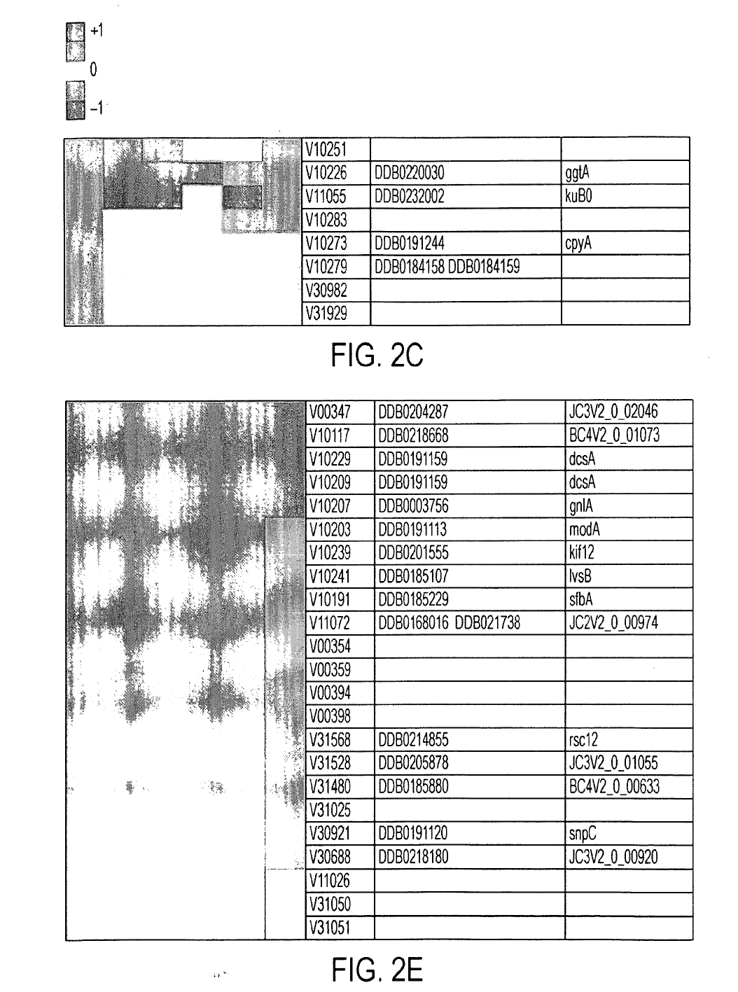

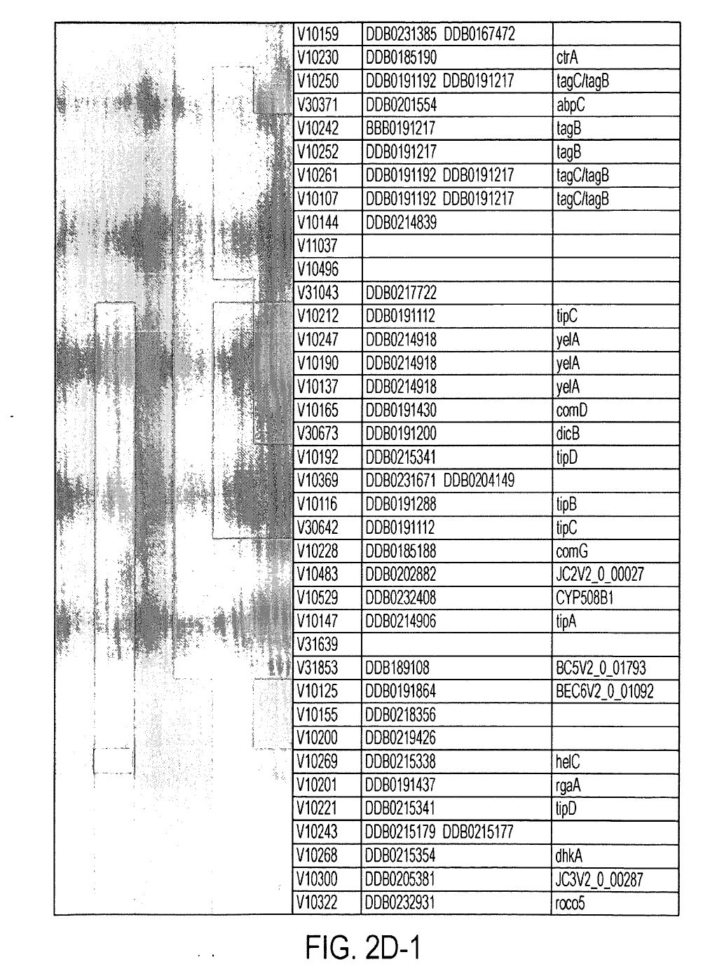

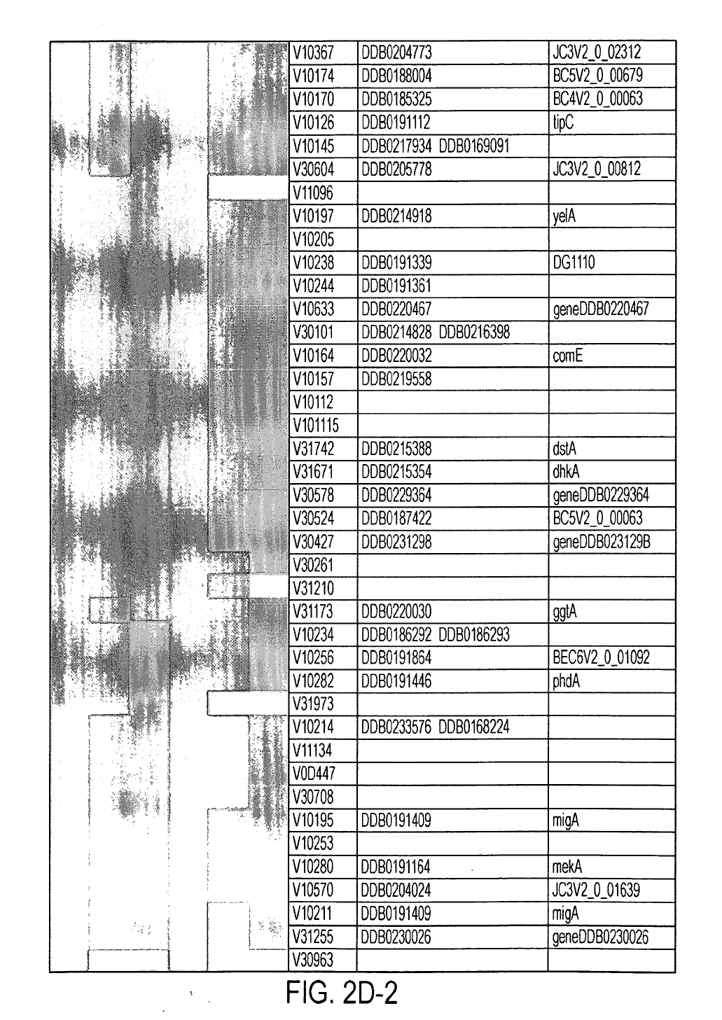

In FIG. 2a, the mutants are categorized based on the phenotype matrix using a hierarchical clustering method. Eisen, M. B., Spellman, P. T., Brown, P. O. & Botstein, D., Cluster analysis and display of genome-wide expression patterns, Proc Natl Acad Sci U S A 95, 14863-8 (1998). One observed result is that 83% of the total number of mutant clones (1870 of 2257) cannot be distinguished from wild type (blue-green in FIG. 2a), possibly because the insertion is in an intergenic region, or the mutated gene exists redundantly or is nonessential for growth and development under the present condition. The second noticeable feature of these data is the number of strains clustered at the bottom of FIG. 2a (139 clones appearing with two or more yellow boxes) and sparsely distributed elsewhere. Many of these exhibited slow axenic growth and the low cell-density effect associated with it despite multiple attempts to grow them. This phenotype may be largely due to a systematic bias carried over from the parent, since most of them are from the same transformant set.

After removing these systematic aberrations, it is estimated that 1 to 2% are defective in genes that, while permitting vegetative growth on bacteria, interfere with normal growth in axenic medium. For several mutants in this category, the observed behavior was independently confirmed by disrupting the gene using homologous recombination. The third feature of this data set is the remaining strains with developmental phenotypes, representing 4% of the clones in the unbiased mutant set (76 strains out of 1799) and 32% in the prescreened mutant set.

Thus, mutant strains that may be used according to the invention may have the following characteristics: they exhibit vegetative growth on bacteria, but have developmentally aberrant phenotypes, such as early wave mutants, pulse mutants, slow oscillator mutants, optical density mutants, PKA pathway mutants, slug mutants, and combinations thereof.

Strains that exhibited almost no development, or aberrant behavior throughout all developmental stages, are clustered at the top of FIG. 2a (expanded in FIG. 2b; N=30). This cluster includes a group of ‘developmentally null’ mutants in which genes such as mApA, piaA, yakA and dagA are disrupted. Other groups include DG1105, DG1037, DG1122 from an earlier screen (Loomis unpublished), as well as another group that includes the PKA pathway genes rdeA and regA). These mutants not only show early developmental defects, but continue to exhibit aberrant behavior until mound formation, and are either stalled or show further aberrant behavior during slug migration and culmination. The above mutant clusters are followed by a cluster consisted of mutants with similarly severe phenotype plus growth stage defects (FIG. 2c).

Another major mutant cluster contains clones showing defective behavior at the slug and culmination stage, but wild type behavior during aggregation (FIG. 2d). Particularly noticeable are five mutants disrupted in tagB/C, mutations in tipA, tipB, tipC and tipD, and multiple occurrence of mutants with insertions in the yela gene and the dhkA gene. At the bottom of FIG. 2d there are strains that show aberrant behavior in the early stage but nevertheless form mounds, but again show deficient slug and fruiting body structure. A large number of mutants defective in early signaling are also defective later in development (FIG. 2d) even though they appear to stream normally to aggregation centers. This suggests either that the gene products are used at two or more different times during development—for example, cAMP metabolism—or that wave phenotype dictates later aspects of morphogenesis. Finally, some strains exhibited aberrant behavior during the early signaling to aggregation stages, but no striking phenotypes during later stages. These strains may be contrasted with those exhibiting defects only at the slug stage or the culmination stage (FIG. 2e), such as those disrupted in the cellulose synthase gene dcsa (FIG. 2e).

Co-clustering of independent clones disrupted in the same genes provides a strong validation of this parallel profiling approach. In general, the developmental stages observed for most of the published mutants examined here agree with the literature. Mutants previously characterized as aggregation minus fail to aggregate, and stalk defective mutants fail to make stalks. Detailed phenotypes, such as the early breakup of aggregation streams seen in erkA and phdA, and long stalks in dhkA also agree well with known mutant phenotypes. This result is not surprising because the data were gathered under well controlled environmental conditions, in a systematic fashion, and with cells first grown in axenic media then plated on agar plates then followed for the entire life cycle.

In some embodiments, wavelet analysis can be used to quantitatively measure the integrated response of the cells. For example, wavelet analysis was performed as described in Sawai et al. with some modification. See Sawai, S., Thomason, P. A. & Cox, E. C., An autoregulatory circuit for long-range self-organization in Dictyostelium cell populations, Nature 433, 323-326 (2005). Briefly, from the original movie files taken using the CCD camera, time-series ρ(x, y; t) of average pixel intensity from 3×3 pixel areas at coordinate (x, y) were sampled from a mesh of 20 pixel intervals (M=2048 sites). From the time-series, normalized wavelet power spectra averaged over space were obtained by using the following formula:

$$\overline{{\vert W ( s , t ) \vert}^{2}} = \frac{1}{M} {\sum }_{x , y} \frac{1}{{σ}_{xy}^{2}} {\vert {\sum }_{{n}^{′} = 0}^{N - 1} ρ ( x , y ; t ) ψ \{ ( {n}^{′} - n ) Δ t / s \} \vert}^{2}$$

where Δt is the time interval of the time series ρ(x, y; t) with variance σ_{xy}², and ψ is the Morlet wavelet:

ψ(η)=π^{-1/4} exp[wω₀η−η²/2]

where ω₀=6. These procedures were automated and integrated with image acquisition. Feature extraction from the wavelet analysis was performed using a script written in Perl that traces the peak of the wavelet power spectrum as a function of time. A running average with a time interval of 6.7 min was employed to remove short time-scale fluctuations. The resulting trace data were used for clustering using a K-mean algorithm.

For each developmental stage-growth, early wave, aggregation, mound, slug and fruiting body-deviation from reproducibly robust wild-type behavior at each stage was noted. This information was ranked into four different categories pᵢⱼ=−1, −½, 0, ½ or 1, where i ∈ [1, 6] stands for ordered developmental stage (e.g. i=1 is the growth stage) and j represents the sample number. Category pᵢⱼ=1 is thus the value for a given wild type phenotype, and pᵢⱼ<1 signifies the severity of the mutant phenotype. pᵢⱼ=−1 corresponds to a null-phenotype, meaning that a developmental stage-specific behavior and morphology was completely absent, either due to developmental arrest at that particular stage, or at a preceding stage. pᵢⱼ=−1 is assigned when a clear deviation from wild type behavior could be identified (e.g. slow oscillations, short stalk, and so on). A phenotypic score of pᵢⱼ=−½ was assigned when the phenotype could not be distinguished from phenotypic fluctuations exhibited from experiment to experiment with wild-type cells.

Many clones (V10546-V10646, V10676-V10696, V30001-V30896, V31301-V31596) systematically showed late slug behavior characterized by loss of cells from the slug posterior and early culmination. These were assigned pᵢⱼ=0 and treated as wild type for clustering purpose. Multiple sample runs were averaged by taking the maximum

$${q}_{sj} = {{max}^{Ns}}_{i = 1} {p}_{ij}$$

for strain s with Ns repeated runs. Although this filtering approach loses some information relative to simple mean averaging, it supplies a more rigorous justification for the claim that a given strain is defective in some aspect of development. Clustering was performed using Cluster 3.0 and Java TreeView. See Sutoh, K., A transformation vector for Dictyostelium discoideum with a new selectable marker bsr, Plasmid 30, 150-154 (1993); Rice, P., Longden, I. & Bleasby, A. EMBOSS: the European Molecular Biology Open Software Suite. Trends Genet 16, 276-7 (2000).

The quantitative methods described previously can also be employed to determine whether a compound tested using the assay systems of the present invention achieves a desired effect, including a desired integrated response. For example, one of the new mutant phenotypes discovered by Sawai et al (2005 Nature) is mutant in a chloride channel. The phenotype is very long-period shallow-amplitude waves. This strain and the described methods could be used in a screen for channel blockers and rectifiers, in the former case screening for compounds that mimic the mutant wave phenotype, in the latter, compounds that restore the WT phenotype. Mutations in Dictyostelium rdeA and regA cannot propagate coherent waves, forming pathological numbers of small spiral waves which are detected as an abundance of spiral cores and abnormal wavelet portraits. These cores are analogous to reentrant waves in heart attacks. Drugs that prevent/ameliorate reentrant waves are important in cardiology and the system described can be used to screen for such drugs by comparing control wave patterns in mutant and wild-type with and without added test compounds.

The above described systems and components for use therein can be used in methods, systems, and kits of the present invention for screening a compound for biological activity using a culture of Dictyostelium cells and an automated image capture device to capture images of the culture and to compare the integrated response of cells with the compound to a control.

In some embodiments, the present invention is directed to kits for screening a compound for biological activity, the kits comprising selected cells or spores of Dictyostelium cells suitable for culturing, e.g., under nutrient deprived conditions, to test a compound using an automated image capture device.

The present invention is also directed to methods for preparing more than one culture of Dictyostelium cells, the method comprising: (a) providing a cell culture of Dictyostelium cells; (b) scaling up the culture of (a) to generate more than one clonal population in parallel; and (c) growing the populations of (b) under nutrient deprived conditions to produce more than one culture of Dictyostelium cells. In some embodiments, over 50, over 100, or over 1000 clonal populations can be generated.

The following examples further illustrate the present invention, but are not to be construed to limit the scope of the present invention.

### Example 1

A culture of Dictyostelium cells can be prepared using various methods known to one of skill in the art. For example, clones of random insertional mutants generated by REMI and wild type Dictyostelium discoideum cells were grown on fresh lawns of Klebsiella aerogenes on SM agar for three to four days. Aljanabi, S. M. & Martinez, I., Universal and rapid salt-extraction of high quality genomic DNA for PCR-based techniques, Nucleic Acids Res 25, 4692-3 (1997). The cells were picked from a feeding-front of a plaque into 2 ml growth medium (PS medium one liter; 10 g Special Peptone (Oxoid), 7 g Yeast Extract (Oxoid), 15 g D-glucose, 0.12 g Na₂HPO₄.7H₂O, 1.4 g KH₂PO₄, 40 μg vitamin B12, 80 μg folic acid) supplemented with 1× Antibotic-Antimycotic (Gibco).

30 clones were cultured in parallel using five 6-well plates (Costar 3506; Corning). After incubation at 22° C. for one day, bacteria and other debris were removed by gentle shaking followed by aspiration of the medium. Fresh PS medium was then added and the cell density was readjusted if necessary. The cells were allowed to attach to the bottom of the plate and incubated at 22° C. for another 24 hrs. Cell density in the initial inoculation was typically 2×10⁶ cells/well. Under these conditions, wild-type AX4 cells attach robustly to the plate surface and appear non-polarized. They grow and divide ˜3 times at a doubling time of approximately 12 hours before reaching confluency at 7×10⁶ cells/well.

Growth medium was then removed and the cells were re-suspended in lml DB (10 mM KH₂PO₄/Na₂HPO₄, 2 mM MgSO₄, 0.2 mM CaCl₂; pH 6.5) and transferred to a 1% agar (Bactoagar; Gibco) surface prepared in 6-well plates where they were allowed to settle for 15 minutes to form a monolayer. Supernatant was removed and the plates were allowed to dry for 15 min in a sterile hood.

### Example 2

The life cycle of 2257 mutagenized clones were analyzed. These were of two major types. To test the generality of our approach, we analyzed a collection of mutants generated by insertional (REMI) mutagenesis, many of which have been published. These strains came from the Loomis and Shaulsky laboratories. They are numbered V00262 to V10300. To test our methods to discover new mutants with developmental phenotypes by unbiased random REMI mutagenesis, we analyzed a subset of an extensive collection developed at Baylor. These are the V10301-V11139 and V30000-V31999 series. Whenever the phenotype deviated from wild type, the time-lapse experiment was repeated with the result that 882 clones were examined more than once. Of these, 357 were repeated two or more times. REMI mutagenesis provides a convenient and relatively unbiased way to conduct genome-wide forward genetic screens, allowing the investigator to rapidly identify the insertion site by plasmid rescue and inverse PCR. The insertion sites for the entire set were determined at Baylor (dictygenome.bcm.tmc.edu/). Those with suspected aberrant phenotypes were re-sequenced at Princeton on a strain-by-strain basis.

### Example 3

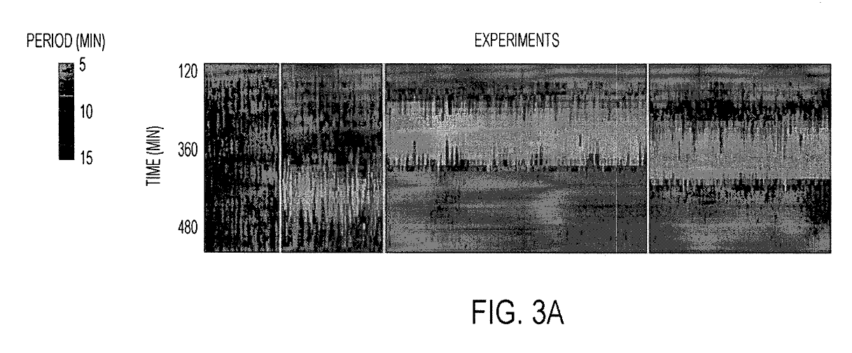

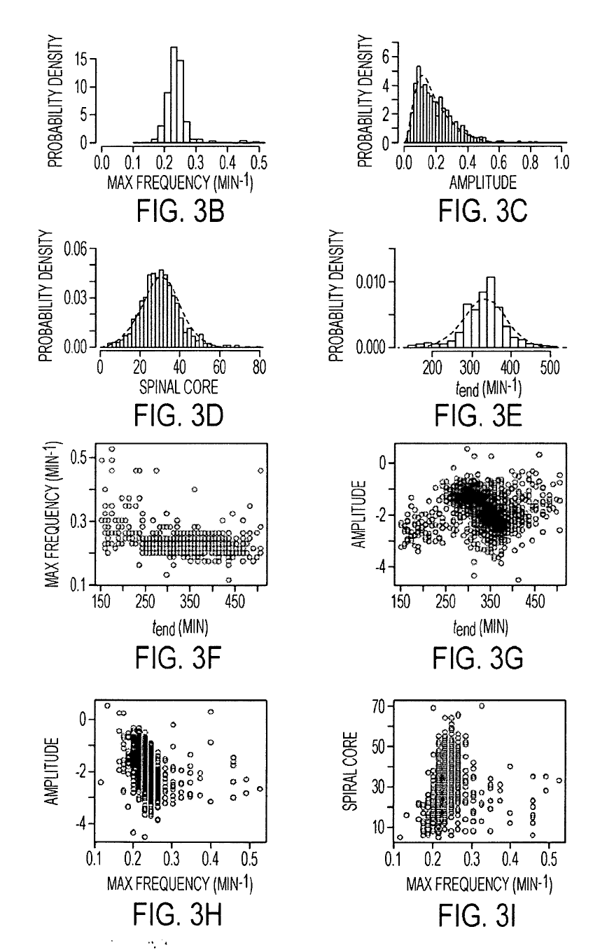

An early wave phenotype mutant can be determined by examining wavelet parameters characterizing early cAMP signaling. For example, the peak of the averaged wavelet power spectrum was traced, and the time of the cessation of signaling tend was determined. The resulting 1-dimensional data can be clustered, yielding a group of samples that failed to exhibit normal oscillation patterns (FIG. 3a; see the next section). At t=t_{end}, the frequency 1/s* and the peak wavelet power spectrum was extracted. FIG. 3b records the distribution of maximum frequency 1/s* of the optical density oscillations. The maximum frequency is narrowly distributed, with an average of 0.24 min⁻¹ (stdev±0.03). This is equivalent to cells reaching approximately a 4.2 min period oscillation, in agreement with previous studies. See Alcantara, F. & Monk, M., Signal propagation during aggregation in the slime mould Dictyostelium discoideum, J Gen. Microbiol. 85, 321-334 (1974); Gross, J. D., Peacey, M. J. & Trevan, D. J., Signal emission and signal propagation during early aggregation in Dictyostelium discoideum, J. Cell Sci. 22, 645-656 (1976); Durston, A. J., The control of morphogenesis in Dictyostelium discoideum in Eucaryotic microbes as model developmental systems (eds. O'Day, D. H. & Horgen, P. A.) 294-321 (M. Dekker, New York, 1977). Compared to the tight distribution of signal frequencies, the wavelet power spectrum follows a log-normal distribution, with mean 0.197 (stdev±0.132) (FIG. 3c). Cessation of oscillations t_{end} is well fitted by a Gaussian distribution (FIG. 3e). A tight frequency distribution and a broad (log-normal) amplitude distribution have also been reported recently in the p53 system and may be a widespread feature of non-linear oscillations in cells. See Geva-Zatorsky, N. et al., Oscillations and variability in the p53 system, Mol Syst Biol 2, 2006 0033 (2006).

The number of spiral wave cores, which is a good measure of the number of cell territories that will later form, also follows a Gaussian distribution (FIG. 3d). This distribution can be explained by the fact that core formation is intrinsically stochastic in nature. Sawai, S., Thomason, P. A. & Cox, E. C., An autoregulatory circuit for long-range self-organization in Dictyostelium cell populations, Nature 433, 323-326 (2005); Palsson, E. & Cox, E. C., On the origin of spiral waves in aggregating Dictyostelium found in: Dictyostelium—A model system for cell and developmental biology. (eds. Maeda, Y., Inouye, K. & Takeuchi, I.) 411-423 (Universal Academy Press, Tokyo, Japan, 1997). It is also likely that the observed distribution depends on sample to sample variability in cell density which may correlate with the oscillation frequency (see below), although the number of aggregation centers is known to be relatively insensitive to cell density above 400 cells/mm² (See Sussman, M. & Noel, E., An analysis of the aggregation stage in the development of the slime molds, Dictyosteliaceae. I. the populational distribution of the capacity to initiate aggregation. Biol. Bull. 103, 259-268 (1952)), and the experiments described herein were carried out at around 7000 cells/mm². To exclude such complications, the data in FIG. 3b-i were obtained from selected samples exhibiting spiral wave propagation where the growing cells had reached confluency and showed no growth defects (N=1639). It has been observed that the number of aggregates exceeds the number of spiral cores because streams tend to break up just before aggregation completes. The extent of late stream breakup was highly variable from a sample to sample, even for the same strain, and therefore this phenotype was not considered as a robust trait for further annotation.

FIG. 3f-i displays these data as scatter plots. The following has been observed: first, when the system develops quickly, there is a weak tendency for the oscillation frequency to be smaller (FIG. 3f). Second, there appears to be a weak positive correlation between the amplitude and t_{end} (FIG. 3g) and a negative correlation between the amplitude and the frequency at t_{end} (FIG. 3h). Heterogeneity in the signaling response has been reported at the single cell level. Dormann, D., Weijer, G., Parent, C. A., Devreotes, P. N. & Weijer, C. J., Visualizing PI3 kinase-mediated cell-cell signaling during Dictyostelium development, Curr. Biol. 12, 1178-1188 (2002); Samadani, A., Mettetal, J. & van Oudenaarden, A Cellular asymmetry and individuality in directional sensing, Proc Natl Acad Sci U S A 103, 11549-54 (2006). Since the analysis is based on data from groups of cells, wave amplitude measures the coherence among the cells of the periodic cytoskeletal rearrangement upon cAMP stimulations. The data, therefore, suggest that the cells are participating in the wave signaling more heterogeneously when the system takes a shorter time to reach the streaming stage, and/or when it reaches a high frequency oscillation state. Formation of spiral cores depends on how excitability evolves in time. Sawai, S., Thomason, P. A. & Cox, E. C., An autoregulatory circuit for long-range self-organization in Dictyostelium cell populations, Nature 433, 323-326 (2005). In high frequency samples, more spiral cores are observed (FIG. 3i). From the slope, there is roughly a five-fold increase in maximal number of spiral cores as the frequency increase from 0.17 min⁻¹ to 0.25 min⁻¹. The data suggest a causal relationship between the formation of spiral cores and heterogeneity in cell excitability.

### Example 4

Identifying pulsing or slow-oscillator mutants can be done by first placing sample runs into four groups using K-mean clustering of the wavelet transform (FIG. 3a), then removing possible pleiotropic effects during the growth phase by cross-verification with the phenotype cluster. The first two clusters contain samples with slight differences in the onset that is within that observed in the wild type. The third cluster in FIG. 3a, with delayed wave onset, contains mostly low density samples, while samples in the last cluster failed to establish wild type waves.

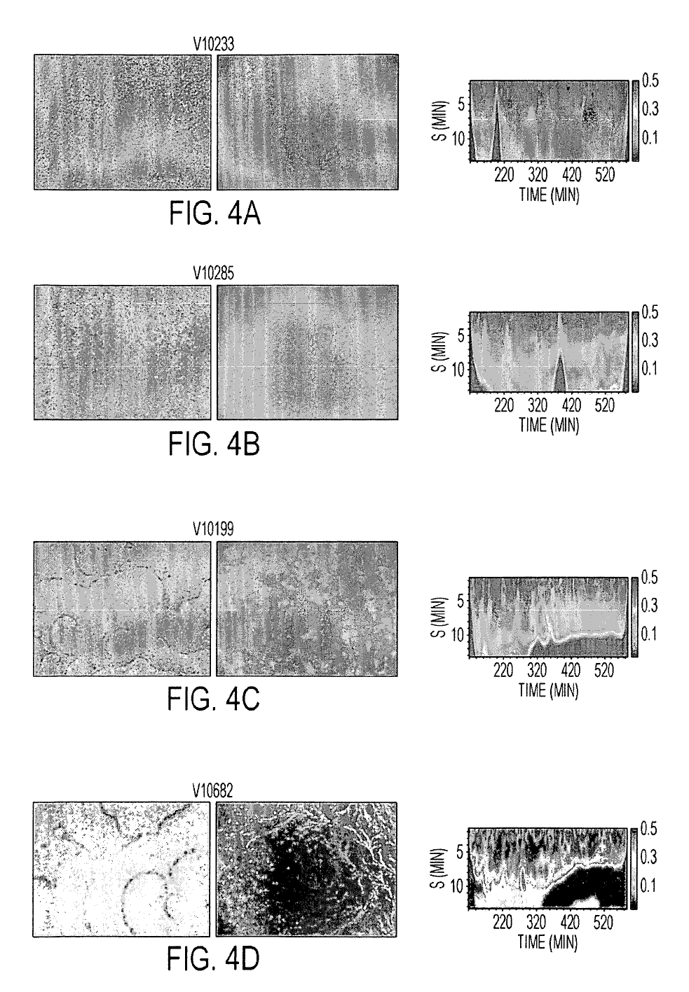

The mutants detected this way display a range of severity in signaling defect. For example, V10233 is disrupted in the piaA gene (FIG. 4a), encoding a TOR (Target of Rapamycin) Complex protein that is required for the cAMP pulse-induced activation of adenylyl cyclase. Lee, S. et al,. TOR complex 2 integrates cell movement during chemotaxis and signal relay in Dictyostelium, Mol Biol Cell 16, 4572-83 (2005). Neither optical density waves nor signs of aggregation are visible, as expected from the known null phenotype of piaA mutants. V10285 (DG1105; dictyBase ID: DDB0220018) shows local pulsatile waves, and development at this stage is prolonged (FIG. 4b). V10199 (DG1037; dictyBase ID: DDB0191301) shows slow oscillations of extended duration (FIG. 4c), and development appears to be arrested during early aggregation. Due to the long periodicity of the optical density oscillations, the wavelength of the spirals is extended, and therefore only a few spiral wave territories appear. Finally, V10682 is able to develop after growth and starvation on bacterial plates, but on non-nutrient agar development is delayed from early aggregation on (FIG. 4d). Optical-density wave onset is late, and wave periodicity remains long and never reaches the characteristic 5 min oscillation. The gene disrupted in this strain (dictyBase ID: DDB0218077) encodes a protein homologous to the conserved clc6/7 type chloride channel family protein.

### Example 5

Two strains (V10258 and V30230) that exhibit notably altered wave and aggregation phenotype (FIGS. 5b,c) are found together in the clustered array (FIG. 2b). In these mutants, waves propagate for very short distances before annihilating when they crash into each other. Compared to wild type behavior (FIG. 5a), periodic signaling begins early in both strains, and the signaling duration is abbreviated to 1 hr (FIG. 5; purple bar right panels). Cells aggregate precociously, forming small mounds with very little evidence of streaming toward a spiral center. Furthermore, the aggregation process is completed in 3 hr. These features are clearly seen in the wavelet analysis (FIG. 5 right panels).

Strain V30230 and V10258 carry an insertion in the regA gene and the rdeA gene, respectively. The regA gene encodes an intracellular cAMP phosphodiesterase with a response regulator domain at the N-terminus (Thomason, P. A., Traynor, D., Stock, J. B. & Kay, R. R., The RdeA-RegA system, a eukaryotic phospho-relay controlling cAMP breakdown, J. Biol. Chem. 274, 27379-27384 (1999); Thomason, P. A., Sawai, S., Stock, J. B. & Cox, E. C., The histidine kinase homologue DhkK/Sombrero controls morphogenesis in Dictyostelium, Dev Biol 292, 358-70 (2006)), and the rdeA gene encodes the only known histidine phosphotransfer domain protein in Dictyostelium discoideum. A biochemical study has shown directly that a receiver domain of RdeA relays phosphate groups to the N-terminal response regulator domain of RegA and that phosphodiesterase activity of RegA is stimulated by phosphorylation of the N-terminal receiver domain. Thomason, P. A., Sawai, S., Stock, J. B. & Cox, E. C., The histidine kinase homologue DhkK/Sombrero controls morphogenesis in Dictyostelium, Dev Biol 292, 358-70 (2006).

It has been shown that PKA pathway mutants can show similar crowded wave phenotypes due to the emergence of abnormally large numbers of spiral cores, and thus this independent isolation of insertions in rdeA and regA is an important confirmation of a recent model of pattern formation that incorporates coupling of external cAMP oscillations to internal cAMP levels. Sawai, S., Thomason, P. A. & Cox, E. C., An autoregulatory circuit for long-range self-organization in Dictyostelium cell populations, Nature 433, 323-326 (2005). Other genotyped mutants related to this pathway were those with insertions in dhkA, dhkc, dhkj and acrA. Mutants in dhkC (V10588) show early slow waves reminiscent of other previously studied PKA pathway mutants pkaR⁻ (Sawai, S., Thomason, P. A. & Cox, E. C., An autoregulatory circuit for long-range self-organization in Dictyostelium cell populations, Nature 433 323-326 (2005)) or dhkK(D1125N) (Miura, K. & Siegert, F., Light affects cAMP signaling and cell movement activity in Dictyostelium discoideum, Proc. Natl. Acad. Sci. USA 97, 2111-2116 (2000)). In contrast, dhkA and acrA show mutant phenotypes only at later stages consistent with their specific roles during slug to culmination stage. A mutant in dhkJ was found in the wild type cluster.

### Example 6

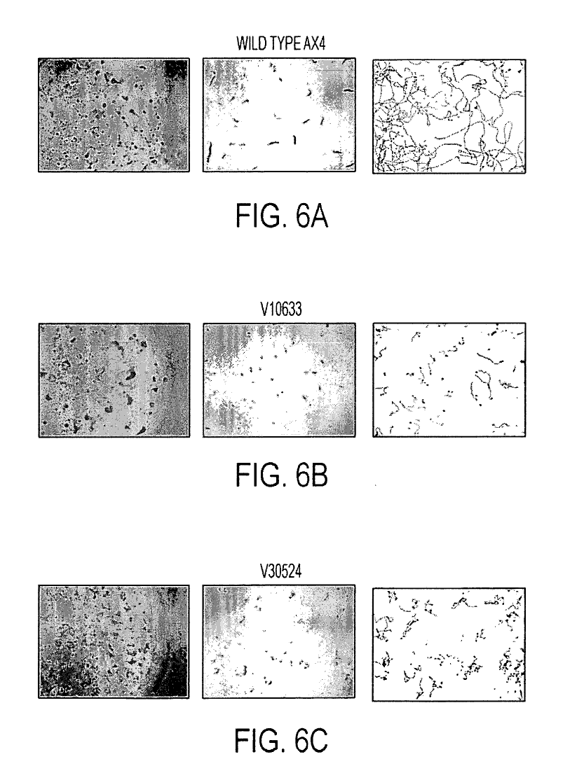

Our dynamical profiling approach reveals mutants with coordination defects. A mutant V10633 of a putative GATA activator (dictyBase ID: DDB0220467) forms chubby slugs that are mostly developmentally arrested at this stage (FIG. 6b, right panel). Migration is almost absent as is evident from the slug trajectories (FIG. 6b bottom). Some slugs do culminate to form fruiting bodies with small spore heads. The video records allow one to discriminate mutants with such behavior from those that proceed to the slug stage but show deficient migration. In V30524, the slugs move with less path persistence compared to wild type (FIG. 6a). V30524 carries an insertion in an open reading frame (dictyBase ID: DDB0187422) that encodes an arginine-N-methyltransferase, a conserved PRMT5 family protein involved in post-translational modification of proteins involved in RNA processing, DNA repair and transcriptional regulation. Wilkins, A. et al., The Dictyostelium genome encodes numerous RasGEFs with multiple biological roles, Genome Biol 6, R68 (2005).

### Example 7

We have demonstrated that parallel phenotyping in a screen based on macroscopic multicellular dynamical features of over 2,000 clonal populations is possible in a relatively short time by combining parallel cell culture, automated high-throughput time-lapse imaging, and quantitative and qualitative phenotyping of multicellular behavior. The time-lapse movies contain a wealth of information that reflects the ability of individual cells to attach to the substratum, signal to one another, perform directional movement towards an attractant, form a multicellular body, migrate as a whole, and differentiate to construct the final culminant. See Supplemental videos to Sawai et al., An autoregulatory circuit for long-range self-organization in Dictyostelium cell populations, Nature 433:323-326 (2005).

The present invention achieves a comparative assay of mutant phenotype under uniform environmental conditions. We demonstrated that mutants disrupted either in the same gene, genes in a common signal transduction pathway or genes known to cause similar morphological defect such as tip mutants can be clustered solely based on a Boolean matrix of the affected developmental stage without any reference to the specific defects observed. The number of major mutant cluster categories was on the order of the number of developmental stages Nᵢ. Assuming random insertion in the mutagenesis, the expected number of developmental genes in each cluster (N_{g}) is approximately N_{g}=(G×P)÷(Nᵢ×r) where G is the number of genes in the genome, P is the mutant frequency and r is the frequency of the coding regions in the genome. We found P=0.04 which is larger than an estimate of 0.3-1% of the clones exhibiting visible developmental aberrations (Eichinger, L. et al., The genome of the social amoeba Dictyostelium discoideum. Nature 435, 43-57 (2005)), suggesting increased sensitivity of mutant detection by our current scheme. Substituting the predicted number of genes in the genome (see Loomis, W. F., The number of developmental genes in Dictyostelium, Birth Defects: Original Article Series 14 no. 2, 497-505 (1978)) (G≅1.25×10⁴; r=0.7) we estimate a total of 720 genes which when disrupted should exhibit a mutant phenotype during development under our assay, and roughly 120 genes on average should constitute a major mutant cluster. This is in line with an estimate of 100-150 genes essential for early development. Amsterdam, A. et al., Identification of 315 genes essential for early zebrafish development, Proc Natl Acad Sci U S A 101, 12792-7 (2004). Our total estimate of developmental genes in Dictyostelium is double the earlier estimate of 300 genes (Amsterdam, A. et al., Identification of 315 genes essential for early zebrafish development, Proc Natl Acad Sci U S A 101, 12792-7 (2004)), and about half of that reported to affect zebrafish morphogenesis (Bonner, J. T. The origins of multicellularity. Integrative Biol. 1, 27-36 (1998)).

### Example 8

Axenic cell culture was scaled up to systematically follow the growth and development of as many as a hundred Dictyostelium clonal populations in parallel (FIG. 1a). Image capture using a robotic system (FIG. 1b) began approximately two hours from the time of nutrient deprivation. FIG. 1c summarizes a typical experiment with our wild type strain, AX4. Cell-cell signaling mediated by extracellular cAMP is visualized by detecting optical density fluctuations that reflect cell shape change in response to passing cAMP waves. Alcantara, F. & Monk, M., Signal propagation during aggregation in the slime mould Dictyostelium discoideum, J. Gen. Microbiol. 85, 321-334 (1974); Siegert, F. & Weijer, C., Digital image processing of optical density wave propagation in Dictyostelium discoideum and analysis of the effects of caffeine and ammonia, J. Cell Sci. 93, 325-335 (1989); Devreotes, P. N., Potel, M. J. & MacKay, S. A., Quantitative analysis of cyclic AMP waves mediating aggregation in Dictyostelium discoideum, Dev. Biol. 96, 405-415 (1983). By 3 hrs a few fragments of weak optical-density waves have begun to emerge from the background. During the next few hours, cells show little directed movement and the cell density is spatially uniform (FIG. 1c, 5 hr). The images are enhanced here by subtracting consecutive frames (FIG. 1c, 3 hr and 5 hr frames, right panels compared to the left). Spiral cores become organizing centers for cell territories by 6.5 hrs, when territories of different sizes with aggregating streams of cells are readily apparent. By 15 hr these territories have become rounded masses of cells, the majority of which by 18 hr have reached the motile slug stage, each slug containing from a few thousand to ˜10⁵ cells. By ˜40 hr the slugs have culminated to form fruiting bodies.

The entire video clip from the first stage of our analysis can be summarized by wavelet analysis, where wave frequency and power spectrum are plotted as a function of time. See Sawai, S., Thomason, P. A. & Cox, E. C., An autoregulatory circuit for long-range self-organization in Dictyostelium cell populations, Nature 433, 323-326 (2005). A typical analysis with wild type cells is illustrated in FIG. 1d. At t=150 min, long-period (15 min) features have begun to emerge. The wave period evolves slowly and smoothly to t=275 min, levels off for 50 min, then abruptly switches off as cells migrate to form well-defined territories. At approximately t=400 min a second long-period feature emerges, corresponding to the cell streaming pattern seen in FIG. 1c at 6.5 hr (see also FIG. 5a). These results are in good agreement with observations on wild type cells grown under conventional culture conditions (see Sawai, S., Thomason, P. A. & Cox, E. C., An autoregulatory circuit for long-range self-organization in Dictyostelium cell populations. Nature 433, 323-326 (2005)), and provide us with a quantitative summary of the first 12 hours of development.

### Example 9

There are many ways in which abnormal cardiac rhythms can lead to heart failure. For example, one very common one, caused by reentrant waves (to use the cardiology language), is caused by a transition first from a Ca++ electrical wave that spreads with circular symmetry from the pacemaker through the heart tissue, synchronizing each heart beat with ventricular contraction. Reentrant waves break this symmetry, causing spiral cores and waves to form. With time, if not treated, more cores form and the entire heart begins to quiver as the spiral core density increases over the surface of the heart. This is lethal if not controlled, usually by defibrillation, since the heart chambers no longer fill and empty periodically. This is analogous to what happens in e.g. a regA mutant of Dictyostelium—the entire monolayer of cells “quivers” and cannot propagate waves because the density of spiral cores has increased many fold over wild type cells.

The present invention can screen for drugs that restore the normal wave pattern in Dictyostelium mutants, or alter the wave pattern in wild type Dictyostelium. With Dictyostelium cells as a test system one can perturb normal wave formation with well known Ca++ blockers, then use this treated system to screen for drugs that restore normal wavelet patterns and spiral core density using the same analysis used for cAMP waves described previously, thus bypassing the expensive, tedious, and uncertain tests in whole animals which is the current standard. This system can also be optionally performed in cardiac cells or in combination with screens of cardiac cells.

In one embodiment cardiac cells from neonatal rats are plated in Hanks or other suitable medium at 3×10̂3 cells/mm̂2 and allowed to form monolayers, at which time they begin to beat coherently and rhythmically, resulting in the propagation of dark-field waves. Control cells receive aliquots of the diluent used to formulate test compounds. Experimental cells receive aliquotes of the test compound diluted serially over an appropriate range. Wavelets and core analyses of the video clips from experimental wells are compared to control wells. The criteria for hits is the modification of wave geometry, particularly the absence or reduction of spiral cores when a test compound e.g. digitoxin is applied to cultures where numerous spiral cores have been provoked by the addition of Ca++ chelators such as EGTA. In another embodiment, wild-type Dictyostelium cells are cultured under conditions where spiral cores are abundant, e.g. asynchronously starved cultures, or synchronously starved cultures seeded with asynchronous cells, and test compounds are added according to the standard protocol described above. Hits are those compounds that diminish in frequency or block entirely spiral waves by restoring circular waves characteristic of synchronous cultures.

### Example 10

Cells of one or more strains of Dictyostelium are placed in growth medium in the wells of a multi-well high throughput assay plate, e.g. a 96 well plate, a 384 well plate, or 16 six-well plates grouped in the canonical 8×12 format. The cells are grown to n 10̂6 cells/well.

A library of test compounds is obtained, and individual compounds are added to individual wells. Some wells are not contacted with a test compound, and are used as controls. Some wells may be contacted with more than one test compound, to measure interactions. The array may include multiple wells of a single phenotypic or genotypic strain of Dictyostelium, e.g., wild type, or a particular mutant.

Alternatively the array may include more than one phenotypic or genotypic strain, arranged in a suitable pattern in the wells, e.g., by column or by row. Because the inventive method involves robotic plate handling and optical equipment, the cultures and test compounds can be arranged in any predetermined pattern in the well plates, if the location of the particular strains and/or test compounds is properly coded into the automated equipment handling system and data analysis software.

In a particular example, strain AX 4 cells are plated into each well of six 16-well plates. One column of 8 wells is left to grow as a control. Each of the other rows is contacted with a test compound, at 8 different concentrations. A titration of concentrations can be 1 nM, 10 nM, 100 nM, 1 μM, 10 μM, 100 μM, 1 mM. Other suitable experimental protocols for high-throughput screening of test compounds will be apparent to a person of ordinary skill.

The plate is placed into an apparatus according to the invention and time-lapse images are captured as described above, one well at a time, with the individual images aggregated into a time lapse video file for each well. The images are processed using software according to the invention, including, e.g., wavelet analysis. The image data is used to characterize the behavior of the cells in culture, including the integrated response, for each well containing cells and/or the control wells. The behavior is compared to test controls and/or predetermined test data and the integrated response of the cells in the culture is determined. The integrated response may be that the cell did not respond to the compound, or it may provide evidence that the test compound modulates a particular receptor, e.g., as an agonist or antagonist.

### Example 11

The following data transformation methods can also be used to quantitatively measure the integrated response of the Dictyostelium cells.

The wavelet analysis can be performed as follows: time series data (500 time points, corresponding to the number of frames in the video) were sampled from 3×3 pixel regions spaced at 20 pixel intervals, for 768 sample points/frame using the ImageJ (National Institutes of Health, USA, available at rsb.info.nih.gov/ij). For each time series, a wavelet function

$${W}_{n} ( s ) = {\sum }_{{n}^{′} = 0}^{N - 1} {x}_{{n}^{′}} ψ * [ \frac{( {n}^{′} - n ) δ t}{s} ]$$

$$ψ ( η ) ∝ {π}^{- 1 / 4} {e}^{i {ω}_{0} η} {e}^{- {η}^{2} / 2}$$

was computed by making use of a convolution theorem and a fast Fourier transform. Here, xₙ is the original time sequence in the δt time interval, and s is the periodicity variable. ψ is a Morlet function (ω₀=6). Wₙ(s) was normalized by mean variance and displayed as a contour plot as a function of (t, s) where t=nδt.

The detection of spatial phase singularities can be performed as follows: the original grey-scale movie data were subtracted between consecutive frames to remove background, reduced in half by averaging and smoothed spatially with a Gaussian filter. The image was then used to obtain time series data from 3×3 pixel areas that were further converted using a time delay embedding technique. Devreotes, P. N., Potel, M. J. & MacKay, S. A., Quantitative analysis of cyclic AMP waves mediating aggregation in Dictyostelium discoideum, Dev. Biol. 96, 405-415 (1983). The resulting movie contains values for each pixel that uniquely defines its state as an angular variable θ in the embedding space. The time delay used for the embedding was chosen based on the first zero-crossing of the auto-correlation function. Spatial phase singularity was searched by calculating a line integral

$${∮}_{c} ∇ θ ( x , y ; t ) \cdot d r = \{ \begin{matrix}0 \\ \pm 2 π\end{matrix}$$

where the integral path c is a closed loop of 3×3 pixels around a position (x, y). Singular points take a value of +2π or −2π depending on the direction of spiral rotation. Phase singularities were counted by time-averaging the line-integral over 50 min intervals for 24 mm×24 mm regions. All analysis was performed using ImageJ software.

Numerical simulations were performed on a 100×100 mesh or a 150×150 mesh using the explicit Euler method with synchronous updating. Excitability E increases from zero or indicated values to Eₘₐₓ=0.93.

These examples illustrate possible embodiments of the present invention. As one of skill in the art will appreciate, because of the versatility of the compositions, kits, and methods of using the compositions disclosed herein, the compositions, kits, and methods can be used in other similar ways to those described herein. Thus, while the invention has been particularly shown and described with reference to some embodiments thereof, it will be understood by those skilled in the art that they have been presented by way of example only, and not limitation, and various changes in form and details can be made therein without departing from the spirit and scope of the invention. Therefore, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A system for screening test compounds for biological activity, the system comprising:
an array of cultures of Dictyostelium cells;
a material handling device that contacts at least one culture with at least one test compound,
an array handling device that positions the array of cultures at predetermined locations,
an automated image capture device that captures images of the cultures; and
a data processor that processes the images and outputs at least one quantitative measurement of the response of the cultures to the test compound.

2. The system of claim 1, wherein the response comprises a spatiotemporal response.

3. The system of claim 1, wherein processing the response of the cultures comprises comparing quantitative measurements of mutant Dictyostelium cells exposed to the test compound to wild type Dictyostelium cells exposed to the test compound.

4. The system of claim 1, wherein the image capture device comprises a fluorescent microscope.

5. The system of claim 4, wherein the image capture device records intracellular protein localization.

6. The system of claim 1, wherein the image capture device comprises dark-field illumination optics.

7. The system of claim 1, wherein the image capture device stores the images in one or more computer-readable files.

8. The system of claim 7, wherein the files comprise a time-lapse video.

9. The system of claim 1, wherein the culture of Dictyostelium cells is a monolayer.

10. The system of claim 1, wherein the compound is selected from the group consisting of an inorganic molecule, a small organic molecule, a drug, prodrug, antibody, vaccine, nucleotide, polynucleotide, amino acid, peptide, polypeptide, virus, and combinations thereof.

11. A method for screening a plurality of test compounds for biological activity, the method comprising:
preparing an array of cultures of Dictyostelium cells;
contacting cultures with test compounds;
capturing images of the cultures using an automated image capture device;
processing the captured images to quantitate a characteristic of the cultures; and
determining from the quantitative culture characteristic and a control value whether the addition of the test compound resulted in a biological integrated response of the culture.

12. The method of claim 11, wherein the control value is determined by reference to a predetermined value.

13. The method of claim 11, wherein the control value is determined by reference to a culture on the same array.

14. The method of claim 11, wherein the quantitative culture characteristic is a spatiotemporal property.

15. (canceled)

16. (canceled)

17. (canceled)

18. The method of claim 11, wherein the image capture device stores the images in one or more computer-readable files.

19. The method of claim 18, wherein the file comprises a time-lapse video.

20. The method of claim 11, wherein the culture of Dictyostelium cells is a monolayer.

21. (canceled)

22. (canceled)

23. The method of claim 14, wherein the spatial properties are selected from the group consisting of cellular growth patterns, cellular aggregation, fruiting body locations, mound locations, slug locations, wave generation, and combinations thereof.

24. (canceled)

25. The method of claim 14, wherein the temporal properties are selected from a group consisting of cellular growth patterns, cellular movement, cellular signaling, wave generation, wave transmission, wave frequency, fruiting body formation, slug formation, mound formation, cellular phenotypic changes, and combinations thereof.

26. (canceled)

27. (canceled)

28. (canceled)

29. (canceled)

30. (canceled)

31. (canceled)

32. (canceled)

33. (canceled)

34. (canceled)

35. (canceled)

36. A kit for screening a test compound for biological activity, the kit comprising:
Dictyostelium cells or spores, selected from wild type and mutant types having a known integrated response to a predetermined category of compounds, when grown in culture and contacted with a compound in the predetermined category, as detected in an automated image capture device.

37. The kit of claim 36, wherein the Dictyostelium cells or spores have a mutation in a portion of the spatial-temporal regulatory pathway selected from the group consisting of CAR1, CAR2, CAR3, CAR4; small GTPases, G protein heterotimers, rdeA, rega, protein ligands required for signal transduction, erk, and PKA.

38. (canceled)

39. (canceled)

40. (canceled)

41. (canceled)

42. (canceled)
